(19) United States
(12) Patent Application Publication
(10) Pub. No.: US 2008/0228284 A1
(43) Pub. Date: Sep. 18, 2008

(54) Specific Polyphosphazene-Containing Three-Dimensional Bone Support Implants and Methods for Their Use

(75) Inventors: Ulf Fritz, Hirschhorn (DE); Olaf Fritz, Hirschhorn (DE); Thomas A. Gordy, Newnan, GA (US); Goetz Martin Richter, Gaiberg (DE)

Correspondence Address: SUTHERLAND ASBILL & BRENNAN LLP, 999 PEACHTREE STREET, N.E., ATLANTA, GA, 30309, US

(73) Assignee: CELONOVA BIOSCIENCES, INC., Newnan, GA (US)

(21) Appl. No.: 11/923,263

(22) Filed: Oct. 24, 2007

Related U.S. Application Data
(63) Continuation-in-part of application No. 11/328,345, filed on Jan. 9, 2006.
Application No. 11/328,345 is a continuation-in-part of application No. 10/250,985, filed on Aug. 8, 2003.
Application No. 10/250,985 is a continuation-in-part of application No. 11/725,709, filed on Mar. 19, 2007.

(30) Foreign Application Priority Data
Aug. 17, 2001 (DE) 10140522.7
Jan. 11, 2002 (DE) 10100961.5
Jan. 11, 2002 (EP) PCT/EP02/00230
Jan. 23, 2002 (DE) 10202467.7
Aug. 12, 2002 (EP) PCT/EP02/09017

(51) Int. Cl. A61F 2/28; A61K 51/06; A61B 17/56

(52) U.S. Cl. 623/23.62; 424/1.65; 606/94

(57) ABSTRACT

The present invention relates to expandable semi-compliant devices that may be used for the treatment of diseased or injured bone tissues, and methods of using the same. The semi-compliant device of the present invention is inserted into an interior space of a cancellous bone tissue, and is filled with a suitable material to provide internal structural support to the bone. The semi-compliant device may further comprise a polymer or a polymer surface coating of a biocompatible polyphosphazene polymer such as poly[bis-(trifluoroethoxy)phosphazene] or derivatives thereof. Such semi-compliant devices or surface coatings may also act as carriers for medicinal, radiological, or thermal treatments of diseased bone.

### CROSS-REFERENCE TO RELATED APPLICATIONS

This application is a continuation-in-part of U.S. patent application Ser. No. 11/328,345, filed Jan. 9, 2006, which claims the benefit under 35 U.S.C. §119(e) of U.S. Provisional Patent Application No. 60/641,968, filed Jan. 7, 2005, the disclosures of which are incorporated herein by reference in their entireties.

This application is also a continuation-in-part of U.S. patent application Ser. No. 10/250,985, filed Aug. 8, 2003, which is the National Stage Entry and claims the benefit of PCT/EP02/00230, filed Jan. 11, 2002, which claims the priority to DE 10100961.5, filed Jan. 11, 2002, the disclosures of which are incorporated herein by reference in their entireties.

This application is also continuation-in-part of U.S. patent application Ser. No. 11/725,709, filed Mar. 19, 2007, which is a continuation of U.S. patent application Ser. No. 10/486,809, filed Apr. 12, 2004, now abandoned, which is the National Stage Entry and claims the benefit of PCT/EP02/09017, filed Aug. 12, 2002, which claims the priority to DE 10202467.7, filed Jan. 23, 2002, and to DE 10140522.7, filed Aug. 17, 2001, the disclosures of which are incorporated herein by reference in their entireties.

### FIELD OF THE INVENTION

The present invention relates to medical devices and compositions for treating diseased or injured bone, including, for example, compositions that can be used to improve the biological acceptance of the medical device within the host subject.

### BACKGROUND OF THE INVENTION

There is presently known in the art a wide range of treatments for diseased or injured cancellous bone tissues in mammals. Cancellous, or spongy bone, has a trabecular (honeycomb structure) and a high level of porosity relative to cortical bone. The spaces between the trabeculae are filled with red bone marrow containing the blood vessels that nourish spongy bone. Spongy bone is found in bones of the pelvis, ribs, breastbone, vertebrae, skull, and at the ends of the arm and leg bones.

All bones are subject to damage by trauma, disease processes, or fractures, such as, but not limited to, osteoporosis, osteoporotic bone, osteoporotic fractured metaphyseal and epiphyseal bone, osteoporotic vertebral bodies, fractured osteoporotic vertebral bodies, fractures of vertebral bodies due to tumors, especially round cell tumors, avascular necrosis of the epiphyses of long bones, especially avascular necrosis of the proximal femur, distal femur and proximal humerus, defects arising from endocrine conditions, and metastatic tumors. The bones comprising the vertebral spine are particularly difficult to treat due to the complexity of their anatomical structure. Effective treatment of the vertebra is further exacerbated by the proximity of the spinal cord to the nerves emanating therefrom.

Two minimally invasive procedures that have gained popularity in the treatment of fractured or diseased bones, and in particular the vertebra, are percutaneous vertebroplasty and Kyphoplasty. U.S. Pat. No. 6,273,916 describes a method and apparatus for performing vertebroplasty. Vertebroplasty is a procedure wherein a cement-like material, such as polymethylmethacrylate (“PMMA”), is injected under high pressure directly into the vertebral cavity. The cement-like material is permitted to cure, and upon hardening, provides structural support to the affected vertebra.

In Kyphoplasty, a small incision is made in the back. Using fluoroscopic imaging techniques, a surgeon guides a cannula to a desired position, inserts a drill through the cannula, and bores through the cortical wall into the cancellous bone to define a channel within the vertebral body. The drill is removed and a balloon catheter is inserted into the channel. The balloon catheter is then inflated to compress the cancellous bone against the inner cortical wall to define a cavity therein. A particular advantage of this procedure for compression fractures is that inflation of the balloon catheter restores a portion of the vertebral height. Following deflation and removal of the balloon catheter, a cement-like material, such as that used in vertebroplasty, is injected to fill the cavity. The cement is permitted to cure, and the surgical site is closed.

Variations of percutaneous vertebroplasty and Kyphoplasty have been disclosed. For example, U.S. Pat. No. 5,827,289 discloses using a balloon to form or enlarge a cavity or passage in a bone, especially in, but not limited to, vertebral bodies and to deliver therapeutic substances to bone in an improved way. U.S. Pat. No. 6,632,235 discloses using inflatable devices for reducing fractures in bone and treating the spine. U.S. Patent Application Publication No. US 2003-0050644 A1 discloses employing an expandable body that is inserted into bone over a guide wire. U.S. Patent Application Publication No. US 2005-0234456 A1 discloses using an implantable medical device for supporting a structure. U.S. Pat. No. 6,348,055 discloses using a conduit for delivering an implant material from a high pressure applicator to an implant delivery device. U.S. Pat. No. 6,033,411 discloses using precision depth-guided instruments to perform percutaneous implantation of hard tissue implant materials.

While the aforementioned procedures represent significant advances in the treatment of bone injuries and diseases, they are not without risk. A risk common to both procedures is the exfiltration of the cement from a fracture site in the treated bone. While these risks are more pronounced in vertebroplasty, due to the high injection pressures, exfiltration of the cement from the fracture site can lead to thrombosis, spinal stenosis, or nerve root compression, and in rare cases pulmonary embolus.

A further limitation of the aforementioned procedures is that once the bone cement has cured, subsequent removal of the cement from the vertebral body is prohibitive, particularly in the case of vertebra in the spine.

Similarly, the aforementioned methods are reparative and make no provision for the treatment of any underlying disease condition which may have caused or contributed to the fractures necessitating the application of these methods in the first place.

Accordingly, despite these recent advances in the art, there remains a continuing need for improved devices and methods for treating bone fractures and disease conditions.

### BRIEF SUMMARY OF THE INVENTION

The present invention is directed to a method of treating diseased or injured bone tissue comprising selecting an interior area in a bone tissue to be treated, inserting a device into the interior area of the bone tissue to be treated, and internally supporting the bone tissue using the device during treatment.

The present invention is also directed to a device for treating diseased or injured bone comprising a catheter, wherein the catheter comprises a main body defining at least one interior passage therethrough, an expandable semi-compliant structure, wherein the semi-compliant structure defines an interior space, and a removable fastener, wherein the fastener releasably connects the catheter to the semi-compliant structure.

In one aspect of this invention, osteogenesis and bone healing may be facilitated by use of implants coated with or comprising a specific polyphosphazene polymer known as poly[(bistrifluorethoxy)phosphazene or derivatives thereof. Poly[(bistrifluorethoxy)phosphazene] has inherent anti-inflammatory and antibacterial properties and inhibits the accumulation of thrombocytes. Implants for the treatment of diseased or injured bone coated with or otherwise comprising poly[(bistrifluorethoxy)phosphazene] offer improved treatment and outcomes. These and other aspects of the present invention are disclosed in detail below.

### BRIEF DESCRIPTION OF THE SEVERAL VIEWS OF THE DRAWINGS

FIG. 1 is a side elevational view of a detachable semi-compliant structure and catheter according to one embodiment of the invention inserted into a cavity defined in the cancellous bone of a vertebra.

FIG. 2 is a side elevational view of the detachable semi-compliant structure and catheter of FIG. 1 as expanded by a bone-supporting material.

FIG. 3 is a side elevational view of decoupling of the catheter of FIG. 1 from the semi-compliant structure of FIG. 1 and sealing of the semi-compliant structure.

FIG. 4 is a side elevational view of a semi-compliant structure after implantation in a vertebra.

FIG. 5 is a transverse cross-sectional view of a spinal vertebra and arthroscopic probe inserted therein.

FIG. 6 is a transverse cross-sectional view of a spinal vertebra having a semi-compliant structure and arthroscopic probe that is partially inserted into a vertebral body.

FIG. 7 is a transverse cross-sectional view of a spinal vertebra having a semi-compliant structure and arthroscopic probe that is fully inserted into a vertebral body.

FIG. 8 is a schematic representation of a general cryoextraction scheme used to prepare particles according to one embodiment of this invention.

FIG. 9 shows the manual dripping technique by which the polymer solution was supplied to liquid nitrogen in preparation of the microspheres, used in one aspect of this invention.

FIG. 10 is a side elevational view of the detachable semi-compliant structure and catheter of FIG. 1 as expanded by a bone-supporting microspheres comprising a polyphosphazene polymer such as poly[bis(trifluoroethoxy)phosphazene].

### DETAILED DESCRIPTION

The present invention relates to the field of orthopedic surgical devices and techniques. The method of treatment of the present invention, as illustrated in reference to the various drawings, involves using a catheter 67 that is connected to a preferably detachable semi-compliant structure by a removable fastener, preferably a screw device. The fastener releasably connects the catheter 67 to the semi-compliant structure 49 and is capable of coupling the semi-compliant device 49 to the catheter 67 and decoupling the semi-compliant device 49 from the catheter 67.

The catheter 67 has a main body defining at least one interior passage therethrough, the semi-compliant structure defines an interior space, and the semi-compliant structure comprises a sealable port that allows for fluid communication between the interior passage of the catheter and the interior space of the semi-compliant structure.

“Semi-compliant structure” is defined herein as a malleable, expandable, non-rigid structure. This is in contrast to a totally compliant structure, or a rigid, non-compliant structure. Semi-compliant structure more specifically defined as a structure that has a specific compliance rate of about 10% to about 30%. It should be understood that such rate is non-limiting to the scope of the invention. The compliance rate of the semi-compliant structure is defined as the rate at which the structure yields to pressure or force without disruption, or an expression of the measure of the ability to do so, such as an expression of the distensibility of the semi-compliant structure, in terms of unit of volume change per unit of pressure change, when it is filled with liquids or other materials.

The semi-compliant structure may be temporarily or permanently inserted in an interior area such as a cavity or other space within diseased or injured cancellous bone tissue of a mammal in order to internally support the bone and/or to treat such diseases or injuries, and to alleviate symptoms of such diseases or injuries, such as back pain. The detachable semi-compliant structure expands upon introduction, typically by injection, of a suitable bone-supporting material, through a passage within the catheter, and the semi-compliant structure provides containment and maintenance of the bone-supporting material therein. The detachable semi-compliant structure is preferably shaped such that upon expansion, the structure will generally adapt and conform three-dimensionally to the dimensions of the exterior area such as a cavity defined within the internal cortical walls of the bone to be treated. The detachable semi-compliant structure prevents the exfiltration of the bone-supporting material from the fracture site through use of a preferred semi-permeable membrane, and facilitates controlled drainage from the structure, thereby avoiding the deleterious effects described herein above.

To provide additional containment and maintenance of the bone-supporting material within the structure, the structure may be provided with a sealable port, through which the catheter communicates with the semi-compliant structure. The port may be sealed upon detachment of the catheter to prevent the bone-supporting material from exuding from within the structure. This arrangement further facilitates pressurized containment and maintenance of the bone-supporting material within the structure. The port may remain open, but where the bone-supporting material hardens and so cannot exude from the port. In another embodiment, the port may be temporarily sealed so that the catheter can be reattached to the port, and the bone-supporting material can be removed as necessary.

The semi-compliant structure may be formed from any suitable biocompatible material that is malleable and durable, such as, but not limited to, stainless steel, titanium, other metals, metal alloys, polymers including, but not limited to, polymeric materials and plastics such as polyphosphazenes, specifically the polyphosphazene polymer known as poly[bis(trifluoroethoxy) phosphazene] or a derivative of poly[bis(trifluoroethoxy) phosphazene] such as or other alkoxide, halogenated alkoxide, and fluorinated alkoxide substituted analogs thereof, polyester and polyethylene, polylactic acid and copolymers of these polymers with each other and with other monomers, resorbable synthetic materials such as, for example, suture material, Nitinol, or any other suitable material as known to those of skill in the art, including combinations of such materials. In one aspect, the semi-compliant structure is a foil or sheet that provides sufficient malleability. Combinations of such materials include, bur are not limited to, one type of material that may be coated onto a different type of material. The suitable biocompatible material is preferably in the form of a thin film material such as for example a thin metallic film, a polymer film, or a polymer-coated metallic film which is typically super-elastic and possesses excellent rubber-like shape retention. Nitinol, a metal alloy of nickel and titanium, is a particularly suitable biocompatible material because Nitinol has the ability to withstand the corrosive effects of biologic environments, such as that inside cancellous bone tissue. In addition, Nitinol also has excellent wear resistance and shows minimal elevations of nickel in the tissues in contact with nitinol. Betz et al., Spine, 28(20S) Supplement:S255-S265 (Oct. 15, 2003). The use of a suitable Nitinol as a preferred biocompatible material in implantable balloons is disclosed in U.S. Pat. No. 6,733,513, which is incorporated herein by reference.

Nitinol or other biocompatible metallic film materials used in various preferred embodiments of the present invention may further be provided with a polymeric coating of poly[bis(trifluoroethoxy)] phosphazene] or a derivative of poly[bis(trifluoroethoxy) phosphazene], such as other fluorinated alkoxide substituted analogs thereof.

The polymer and polymeric coating of preferred embodiments of the present invention are formed in part of the phosphazene polymer, poly[bis(2,2,2-trifluoroethoxy) phosphazene] or a derivative thereof. Throughout, this polymer is also termed poly[bis(trifluoroethoxy)]phosphazene] and similar names. In one aspect of this invention, the polyphosphazene is poly[bis(2,2,2-trifluoroethoxy) phosphazene] or derivatives thereof, such as other alkoxide, halogenated alkoxide, or fluorinated alkoxide substituted analogs thereof. The preferred poly[bis(trifluoroethoxy) phosphazene] polymer is made up of repeating monomers represented by the formula I shown below:

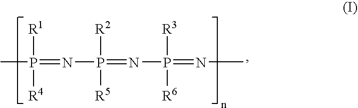

wherein R¹ to R⁶ are all trifluoroethoxy (OCH₂CF₃) groups, and wherein n may vary from at least about 40 to about 100,000, as disclosed herein. Alternatively, one may use derivatives of this polymer in the present invention. The term “derivatives” is meant to refer to polymers made up of monomers having the structure of formula I but where one or more of the R¹ to R⁶ functional group(s) is replaced by a different functional group(s), such as an unsubstituted alkoxide, a halogenated alkoxide, a fluorinated alkoxide, or any combination thereof, or where one or more of the R¹ to R⁶ is replaced by any of the other functional group(s) disclosed herein, but where the biological inertness of the polymer is not substantially altered.

In one aspect of the polyphosphazene of formula (I) illustrated above, for example, at least one of the substituents R¹ to R⁶ can be an unsubstituted alkoxy substituent, such as methoxy (OCH₃), ethoxy (OCH₂CH₃) or n-propoxy (OCH₂CH₂CH₃). In another aspect, for example, at least one of the substituents R¹ to R⁶ is an alkoxy group substituted with at least one fluorine atom. Examples of useful fluorine-substituted alkoxy groups R¹ to R⁶ include, but are not limited to OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF_{3,} OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, and the like. Thus, while trifluoroethoxy (OCH₂CF₃) groups are preferred, these further exemplary functional groups also may be used alone, in combination with trifluoroethoxy, or in combination with each other. In one aspect, examples of especially useful fluorinated alkoxide functional groups that may be used include, but are not limited to, 2,2,3,3,3-pentafluoropropyloxy (OCH₂CF₂CF₃), 2,2,2,2′,2′,2′-hexafluoroisopropyloxy (OCH(CF₃)₂), 2,2,3,3,4,4,4-heptafluorobutyloxy (OCH₂CF₂CF₂CF₃), 3,3,4,4,5,5,6,6,7,7,8,8,8-tridecafluorooctyloxy (OCH₂(CF₂)₇CF₃), 2,2,3,3,-tetrafluoropropyloxy (OCH₂CF₂CHF₂), 2,2,3,3,4,4-hexafluorobutyloxy (OCH₂CF₂CF₂CHF₂), 3,3,4,4,5,5,6,6,7,7,8,8-dodecafluorooctyloxy (OCH₂(CF₂)₇CHF₂), and the like, including combinations thereof.

Further, in some embodiments, 1% or less of the R¹ to R⁶ groups may be alkenoxy groups, a feature that may assist in crosslinking to provide a more elastomeric phosphazene polymer. In this aspect, alkenoxy groups include, but are not limited to, OCH₂CH═CH₂, OCH₂CH₂CH═CH₂, allylphenoxy groups, and the like, including combinations thereof. Also in formula (I) illustrated herein, the residues R¹ to R⁶ are each independently variable and therefore can be the same or different.

By indicating that n can be as large as co in formula I, it is intended to specify values of n that encompass polyphosphazene polymers that can have an average molecular weight of up to about 75 million Daltons. For example, in one aspect, n can vary from at least about 40 to about 100,000. In another aspect, by indicating that n can be as large as ∞ in formula I, it is intended to specify values of n from about 4,000 to about 50,000, more preferably, n is about 7,000 to about 40,000 and most preferably n is about 13,000 to about 30,000.

In another aspect of this invention, the polymer used to prepare the polymers disclosed herein has a molecular weight based on the above formula, which can be a molecular weight of at least about 70,000 g/mol, more preferably at least about 1,000,000 g/mol, and still more preferably a molecular weight of at least about 3×10⁶ g/mol to about 20×10⁶ g/mol. Most preferred are polymers having molecular weights of at least about 10,000,000 g/mol.

In a further aspect of the polyphosphazene formula (I) illustrated herein, n is 2 to ∞, and R¹ to R⁶ are groups which are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen sulfur, phosphorus, or a combination thereof or heteroaryl comprising one or more heteroatoms selected from nitrogen, oxygen, sulfur, phosphorus, or a combination thereof. In this aspect of formula (I), the pendant side groups or moieties (also termed “residues”) R¹ to R⁶ are each independently variable and therefore can be the same or different. Further, R¹ to R⁶ can be substituted or unsubstituted. The alkyl groups or moieties within the alkoxy, alkylsulphonyl, dialkylamino, and other alkyl-containing groups can be, for example, straight or branched chain alkyl groups having from 1 to 20 carbon atoms, typically from 1 to 12 carbon atoms, it being possible for the alkyl groups to be further substituted, for example, by at least one halogen atom, such as a fluorine atom or other functional group such as those noted for the R¹ to R⁶ groups above. By specifying alkyl groups such as propyl or butyl, it is intended to encompass any isomer of the particular alkyl group.

In one aspect, examples of alkoxy groups include, but are not limited to, methoxy, ethoxy, propoxy, and butoxy groups, and the like, which can also be further substituted For example the alkoxy group can be substituted by at least one fluorine atom, with 2,2,2-trifluoroethoxy constituting a useful alkoxy group. In another aspect, one or more of the alkoxy groups contains at least one fluorine atom. Further, the alkoxy group can contain at least two fluorine atoms or the alkoxy group can contain three fluorine atoms. For example, the polyphosphazene that is combined with the silicone can be poly[bis(2,2,2-trifluoroethoxy)phosphazene]. Alkoxy groups of the polymer can also be combinations of the aforementioned embodiments wherein one or more fluorine atoms are present on the polyphosphazene in combination with other groups or atoms.

Examples of alkylsulphonyl substituents include, but are not limited to, methylsulphonyl, ethylsulphonyl, propylsulphonyl, and butylsulphonyl groups. Examples of dialkylamino substituents include, but are not limited to, dimethyl-, diethyl-, dipropyl-, and dibutylamino groups. Again, by specifying alkyl groups such as propyl or butyl, it is intended to encompass any isomer of the particular alkyl group.

Exemplary aryloxy groups include, for example, compounds having one or more aromatic ring systems having at least one oxygen atom, non-oxygenated atom, and/or rings having alkoxy substituents, it being possible for the aryl group to be substituted for example by at least one alkyl or alkoxy substituent defined above. Examples of aryloxy groups include, but are not limited to, phenoxy and naphthoxy groups, and derivatives thereof including, for example, substituted phenoxy and naphthoxy groups.

The heterocycloalkyl group can be, for example, a ring system which contains from 3 to 10 atoms, at least one ring atom being a nitrogen, oxygen, sulfur, phosphorus, or any combination of these heteroatoms. The hetereocycloalkyl group can be substituted, for example, by at least one alkyl or alkoxy substituent as defined above. Examples of heterocycloalkyl groups include, but are not limited to, piperidinyl, piperazinyl, pyrrolidinyl, and morpholinyl groups, and substituted analogs thereof.

The heteroaryl group can be, for example, a compound having one or more aromatic ring systems, at least one ring atom being a nitrogen, an oxygen, a sulfur, a phosphorus, or any combination of these heteroatoms. The heteroaryl group can be substituted for example by at least one alkyl or alkoxy substituent defined above. Examples of heteroaryl groups include, but are not limited to, imidazolyl, thiophene, furane, oxazolyl, pyrrolyl, pyridinyl, pyridinolyl, isoquinolinyl, and quinolinyl groups, and derivatives thereof, such as substituted groups.

Additionally, if desired, polymers other than the poly[bis(trifluoroethoxy) phosphazene] and/or its derivative may be included in the present invention. Examples of polymers may include poly(lactic acid), poly(lactic-co-glycolic acid), poly(caprolactone), polycarbonates, polyamides, polyanhydrides, polyamino acids, polyorthoesters, polyacetals, polycyanoacrylates, and polyurethanes. Other polymers include polyacrylates, ethylene-vinyl acetate co-polymers, acyl substituted cellulose acetates and derivatives thereof, degradable or non-degradable polyurethanes, polystyrenes, polyvinylchloride, polyvinyl fluoride, poly(vinyl imidazole), chlorosulphonated polyolefins, and polyethylene oxide. One may incorporate the selected compounds by any means known in the art, including diffusing, inserting or entrapping the additional compounds in the matrix of an already formed polymeric mass or by adding the additional compound to a polymer melt or to a polymer solvent in the preparation of the polymeric material such as described herein.

In one aspect, the semi-compliant structure may be in the form of an expandable three-dimensional balloon. Where the semi-compliant structure is permanently inserted into cancellous bone tissue, the biocompatible material of the structure is made of a suitable surface material, such as, but not limited to those mentioned above, to provide a bone-friendly membrane for incorporation and healing and to help improve or accelerate the attraction of healthy bone cells.

In applications where disease is the underlying cause of the bone fracture, in one aspect, the present invention further contemplates that the semi-compliant structure serve as a carrier for a treatment for a disease or injury. The invention contemplated herein includes medicinal, radiological and thermal treatments for the underlying disease conditions. Such medical treatments may include, but are not limited to, such treatments comprising drugs such as, but not limited to, Cisplatin, Taxol™, Adriamycin™, Doxorubicin, Melphalan, Cyclophosphamide, Carboplatin, Methotrexate, or similar treatments known to those in the art for treating bone diseases. Such radiological treatments include, but are not limited to, radiation therapy which can be used for treatment of malignant bone disease to prevent further fractures and pain, or interventional procedures which can be applied to malignant bone disease by means of embolization (transvascular occlusion).

The bone-supporting material may include a number of materials that are selected based on the purpose of the treatment. Where the treatment encompasses permanent bone support, the bone-supporting material includes bone cement that may be injected as a liquid and then which hardens within a short period of time. Where the treatment encompasses temporary support of the bone, the bone-supporting material may be injected as a liquid, and will remain a liquid form during the time required for support. It can then be readily withdrawn when the treatment procedure is complete and/or replaced if additional treatment is needed. In alternative embodiments, the bone-supporting material may be in the form of a pliable gel-like material to provide support and energy attenuation for the bone structure.

In yet other alternate embodiments of the present invention, bone-supporting materials as described above may be delivered into the expandable semi-compliant structure of the present invention in association with microspheres or other particles comprising poly[bis(trifluoroethoxy) phosphazene and/or a derivative thereof, provided as microspheres in one or more specified sizes. These particles may further comprise one or more active agents, wherein the active agent(s) may act to retard infection, inflammation, pain, other pathologic conditions, and/or add structural strength and promote osteogenesis in situ. In one aspect of this invention, the polyphosphazene is poly[bis(2,2,2-trifluoroethoxy) phosphazene]. In a further aspect, the polyphosphazene is a derivative of poly[bis(2,2,2-trifluoroethoxy) phosphazene], such as other alkoxide, halogenated alkoxide, or fluorinated alkoxide substituted analogs thereof.

As may be seen in reference to the various drawings, the present invention includes a catheter 67 having at least one lumen or other long extending passage way, preferably a multi-lumen catheter 67, with a detachable semi-compliant structure 49 for temporary or permanent placement in a cavity 74 defined in bone tissue such as cancellous bone tissue 17. The present invention further comprises methods of treating bones which have been fractured through trauma or through disease processes, such as, but not limited to, osteoporosis, osteoporotic fractured metaphyseal and epiphyseal bone, osteoporotic vertebral bodies, fractures of vertebral bodies due to tumors, especially round cell tumors, avascular necrosis of the epiphyses of long bones, especially avascular necrosis of the proximal femur, distal femur and proximal humerus and defects arising from endocrine conditions, metastatic tumors, long bone (i.e., traumatic or spontaneous bone fractures or other local distortions of bone structures), such as cervical, thoracic, lumbar, and sacral fractures, and the like.

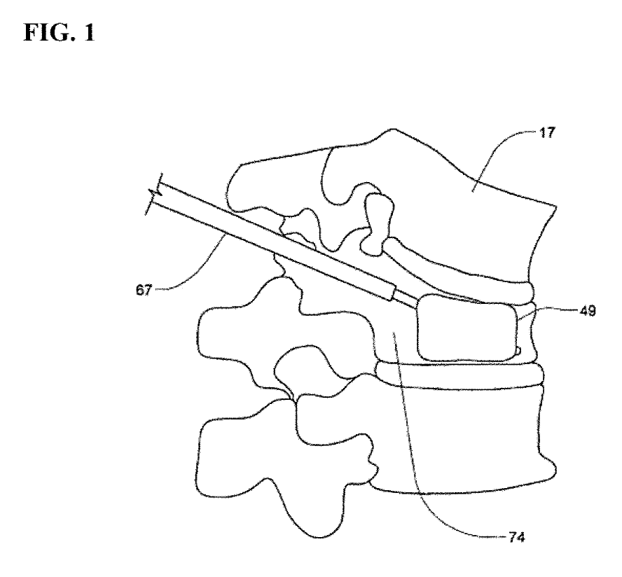

The detachable semi-compliant structure 49, as best shown in FIGS. 1-3 and 10, is shaped such that it generally conforms to dimensions of a cavity 74 selected within the internal cortical walls of the cancellous bone tissue 17. The cavity 74 may be simply identified and/or defined within the internal cortical walls by any suitable procedure familiar to those of skill in the art, such as, but not limited to, drilling, insertion of a precursor inflatable device, and other related methods. The dimensions of the cavity 74 may be predetermined using minimally invasive image-guided techniques such as, but not limited to, X-ray, CT scan or intraoperative CT imaging, ultrasound, computed tomography, MR/CT image registration, three dimensional visualization, optical localization, and magnetic resonance imaging (MRI), or any other suitable imaging techniques. Preferably, the walls of the semi-compliant structure 49 have a compliance rate of about 10% to about 30%, to provide engagement of the structure with the cavity 74 walls comprising either cancellous bone 17 or the internal walls of the cortical bone.

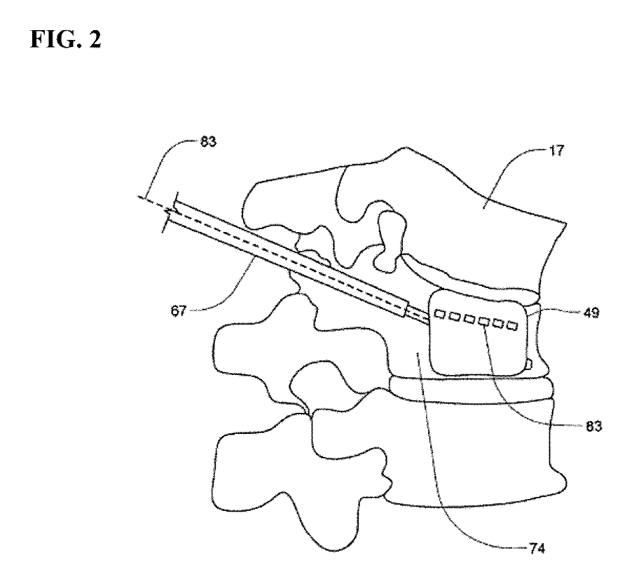

As depicted in FIG. 2, the detachable semi-compliant structure 49 is expandable upon injection of a suitable bone-supporting material 83 through a lumen of the multi-lumen catheter 67, with the structure 49 providing containment and maintenance of the bone-supporting material 83 therein and additional structural support to the cancellous bone tissue 17. The characteristics of the bone-supporting material 83 are selected based upon whether the structure 49 will be a permanent implantation or whether the structure 49 will be temporarily implanted for a sufficient duration to permit a bone fracture to heal.

For permanent implant treatments, the bone-supporting material 83 may be a cement-like material made of a formulation known or to be developed in the art such as those based on polymethylmethacrylate (“PMMA”), or other suitable biomaterial alternatives or combinations, including, but not limited to, dextrans, polyethylene, carbon fibers, polyvinyl alcohol (PVA), or poly(ethylene terephthalate) (PET), such as those used in conventional vertebroplasty or Kyphoplasty procedures. More preferably, the cement-like material is PMMA. Specific formulations of PMMA are known in the art and are commonly used in bone implants. Such formulations include, but are not limited to those disclosed in, for example, U.S. Pat. Nos. 4,526,909 and 6,544,324, which are incorporated herein by reference.

One further aspect of the present invention is to prevent exfiltration of the cement-like material from the fracture site and its resulting physiological risks. This prevention is possible due to the containment and maintenance of the cement-like material within the semi-compliant structure 49.

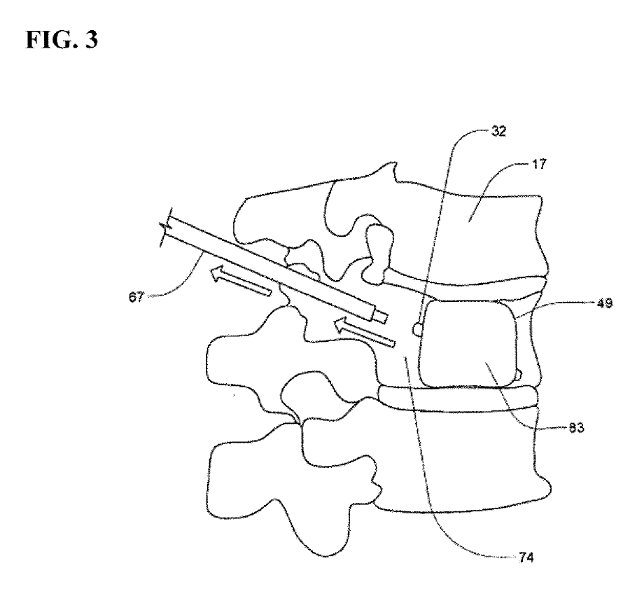

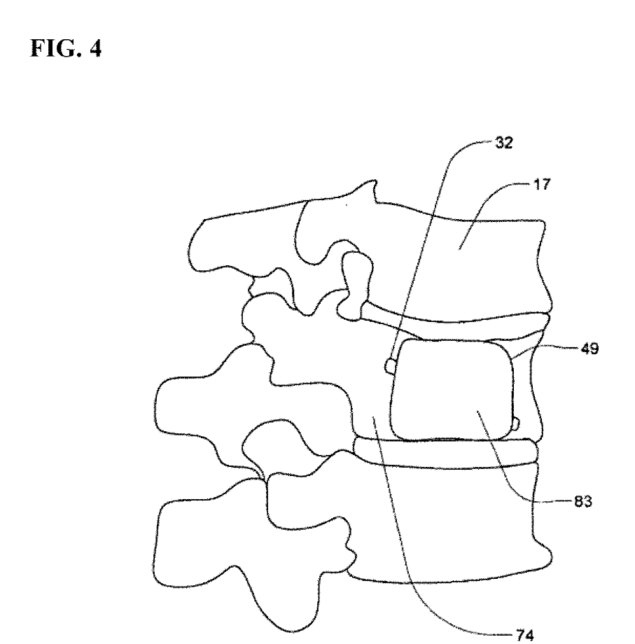

To provide additional containment and maintenance of the bone-supporting material 83 within the semi-compliant structure 49, the structure 49 may be provided with a sealable port 32, as shown in FIGS. 3 and 4, through which the catheter 67 communicates with the semi-compliant structure 49. The port 32 may be sealed upon detachment of the catheter 67 to prevent the bone-supporting material 83 from leaching out of the structure 49. This arrangement further facilitates pressurized containment and maintenance of the bone-supporting material 83 within the structure 49. Additionally, a sealable port 32 also prevents the infiltration of biologic fluids into the semi-compliant structure 49, thereby improving the structure's durability by preventing corrosion and degradation of the walls of the internal semi-compliant structure 49. Alternatively, the catheter 67 may be left attached to the semi-compliant structure 49 until such time as the bone-supporting material 83 has cured. Such curing time generally takes about 2 to about 10 minutes if PMMA is used as the bone cement. Once the PMMA has cured, the catheter 67 may then be detached with minimal risk of the material leaching from the sealable port 32, as shown in FIG. 3. The reverse arrows in FIG. 3 from the bone tissue 17 indicate the direction in which the catheter 67 moves after injection of the bone-supporting material 83 into the semi-compliant structure 49 and decoupling therefrom. However, because this process potentially leaves the structure 49 temporarily open, care should be taken to the extent necessary, to avoid infiltration of the biological fluids into the structure 49.

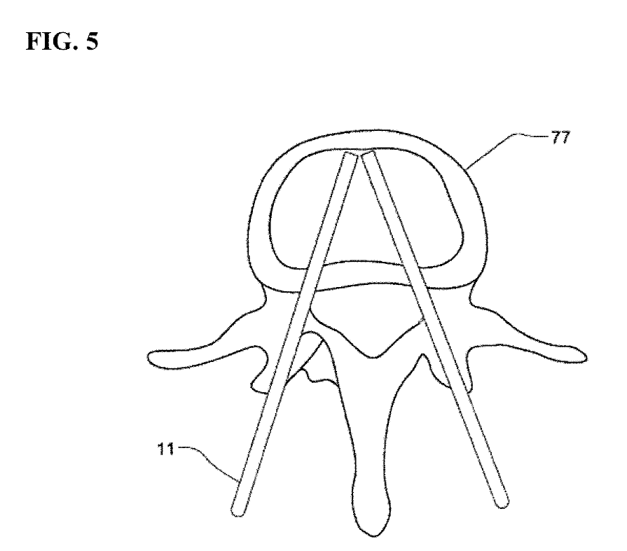

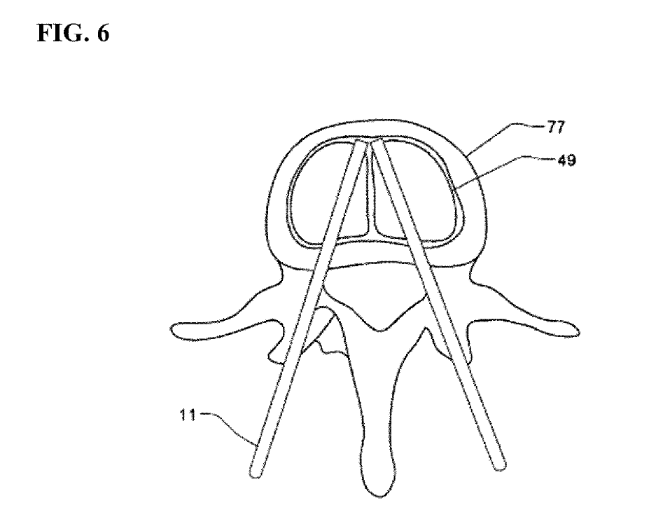

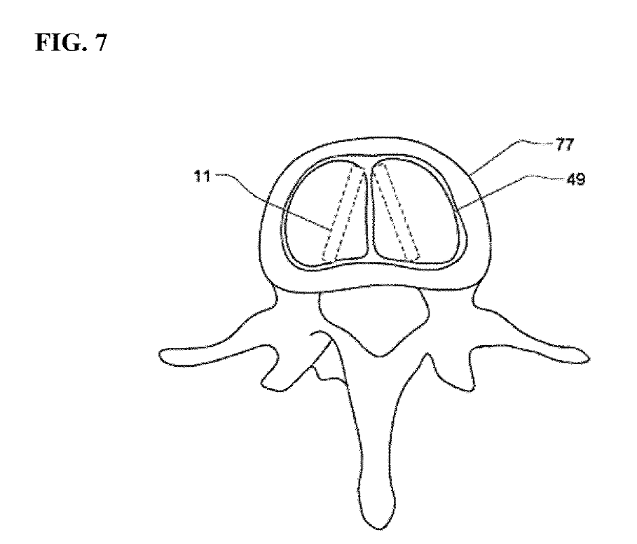

In another aspect, FIGS. 5-7 illustrate transverse cross-sectional views of a spinal vertebra 77 and illustrating an arthroscopic probe 11. In FIG. 5, the transverse cross-sectional view of a spinal vertebra 77 with the arthroscopic probe 11 inserted therein. FIG. 6 illustrates a cross-sectional view of a spinal vertebra 77 having a semi-compliant structure 49 and arthroscopic probe 11 that is partially inserted into a vertebral body, and FIG. 7 illustrates a cross-sectional view of a spinal vertebra 77 having a semi-compliant structure 49 and arthroscopic probe 11 that is fully inserted into a vertebral body.

The device of the present invention may also be utilized for temporary implantation in cancellous bone 17, potentially offering a more advantageous bone setting technique compared to contemporary procedures which rely on insertion of metallic rods, pins or screws to maintain a bone's structure while the fracture is permitted to heal. In this instance the semi-compliant structure 49 would likely require a port having a valve to maintain the strength and rigidity of the structure while the fracture heals, but to allow access to the bone-supporting material 83 for evacuation at a later time. In this instance the sealable port 32 also provides for reattachment of the catheter 67 to permit removal of the bone-supporting material 83 and extrication of the structure from the bone 17.

The characteristics of the bone-supporting material 83 are selected such that it assumes a rigid or semi-rigid state while the bone is healing and is capable of being dissolved, melted, or otherwise withdrawn from the semi-compliant structure 49 once the healing processes have progressed to a point where internal support is no longer necessary. Once the bone-supporting material 83 is evacuated from the semi-compliant structure 49, the structure 49 may then be extricated from the bone to permit final healing of the bone 17. An advantage of the semi-compliant structure 49 over that of metallic rods or pins is that its compliance will facilitate its removal with minimal trauma to the cancellous bone 17 as it is extricated.

The semi-compliant structure 49 may be formed from any suitable biocompatible material, such as, but not limited to, stainless steel, titanium, polymers including but not limited to polyphosphazenes, specifically that polyphosphazene polymer known as poly[bis(trifluoroethoxy) phosphazene] or a derivative of poly[bis(trifluoroethoxy) phosphazene], polymeric materials and plastics such as polyester and polyethylene, polylactic acid and copolymers of these polymers with each other and with other monomers, resorbable synthetic materials such as, for example, suture material, Nitinol, or any other suitable material as known to those of skill in the art, including combinations of such materials. Preferably, the semi-compliant structure 49 will be formed from a biocompatible metallic film material, appropriately shaped to generally conform or adapt to a cavity 74 defined in the internal structure of the bone 17 selected for treatment. An alloy of Nickel and Titanium, commonly known as Nitinol, is well suited to this application, as a result of its proven biocompatibility and its ability to withstand the corrosive effects of biologic environments. Other desirable properties for the metallic film material, and Nitinol in particular, are its super-elasticity and shape memory, which facilitates insertion of the catheter 67 into the cavity 74 defined in the cancellous hone 17. Moreover, Nitinol's stress-strain characteristics make it an excellent choice to provide additional structural support to the bone 17 in combination with the bone-supporting material 83. Nitinol or other biocompatible metallic film materials used in various embodiments of the present invention may further be provided with a polymeric coating of poly[bis(trifluoroethoxy) phosphazene] or a derivative of poly[bis(trifluoroethoxy) phosphazene] or other biocompatible polymers. In addition to titanium, Nitinol, stainless steel, and the like, additional metals and alloys that may be suited to forming the semi-compliant structure 49 disclosed herein include but are not limited to, zirconium, vanadium, niobium, tantalum, chromium, molybdenum, silver, gold, aluminum, silicon, cobalt, palladium, platinum, manganese, combinations thereof, and alloys thereof.

For bone treatments encompassing permanent placement of the structure 49, the biocompatible material is provided with a suitable surface treatment to provide a bone-friendly matrix for incorporation and healing within the cancellous bone 17. In applications where implantation of the structure will be a temporary restorative measure, the surface is prepared to avoid incorporation of and to reduce the adhesion of cancellous bone 17 to the semi-compliant structure 49 thereby facilitating extrication and minimizing trauma to the cancellous bone 17.

Due to the wide range of applications for the semi-compliant structure 49, the bone-supporting material 83 may include a number of materials that are selected based on the underlying purpose of the treatment. Where the treatment is for permanent bone support, the bone-supporting material 83 includes a cement-like material, such as the previously described PMMA formulation, that may be injected as a liquid, paste or gel, and then permitted to cure or harden within a short period of time. Because the cement-like material is contained and maintained within the semi-compliant structure 49, a wider range of cement-like materials is possible, as the material would not encounter the same biochemical environment as faced by uncontained applications.

In instances where the treatment is for the temporary support of the bone 17, the bone-supporting material 83 is injected as a liquid, remains a liquid during the time required for support, and then can be readily withdrawn when the procedure has been completed. In alternative embodiments, the bone-supporting material 83 may be in the form of a pliable gel-like material to provide support and energy attenuation for the bone structure.

In applications where disease is a contributing or underlying cause of the bone fracture, a further aspect of the present invention contemplates that the semi-compliant structure 49 serves as a carrier for treatment of the disease. The aspects of the invention contemplated herein include medicinal, radiological or thermal treatments for the underlying disease condition.

In cases of medicinal treatment regimens, the surface of the semi-compliant structure, which is typically a metallic film material, may be impregnated or coated with a time-release medication targeting the specific disease condition from within the bone itself. Alternatively, the medication may be diffused through a semi-permeable biocompatible material selected for the structure 49 to treat a disease or injury of the bone 17.

In the case of radiological treatment, the radiological treatment is admixed with the bone-supporting material 83 by introducing the admixture into the semi-compliant structure 49, such that it is contained and maintained within the semi-compliant structure 49. In this case, the radiological treatment could be withdrawn from the semi-compliant structure 49, after the appropriate exposure to cancellous bone tissue 17 has been attained. Moreover, as the present invention contemplates temporary implantation of the structure 49, it may also be replaced during radiological treatments or after the completion of all radiological procedures.

The thermal treatment may be provided in the first instance as the bone-supporting material 83 is introduced into the semi-compliant structure 49. The temperature of the bone-supporting material 83 may be adjusted to a desired level prior to introduction into the semi-compliant structure 49. Alternatively, the appropriate temperature may be attained by catalytic reaction of the selected bone-supporting material 83. Re-treatment of the bone tissue 17 may be made by subsequent withdrawal and reintroduction of the selected treatment regimen described herein.

In certain preferred embodiments according to the present invention, the semi-compliant structure 49 may be comprised at least in part of a biocompatible polyphosphazene polymer such as poly[bis(trifluoroethoxy) phosphazene] or derivatives thereof.

In certain other preferred embodiments according to the present invention, the semi-compliant structure 49 may be comprised at least in part of a coating of a biocompatible polyphosphazene polymer such as poly[bis(trifluoroethoxy) phosphazene] or derivatives thereof.

The preferred specific polyphosphazene polymer known as poly[bis(trifluoroethoxy) phosphazene] or a derivative of poly[bis(trifluoroethoxy) phosphazene]. Use of this specific polymer provides surface treatments that are at least in part inorganic in that they include an inorganic polymer backbone and which are also biocompatible in that when introduced into a mammal (including humans and animals), they do not significantly induce a response of the specific or non-specific immune systems. The scope of the invention also includes the use(s) of such surface treatments as controlled drug delivery vehicles.

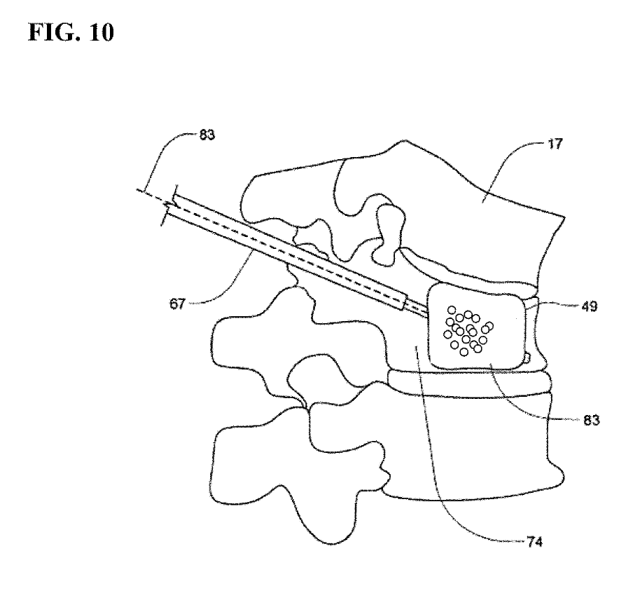

In yet other preferred embodiments of the present invention, particles such as microspheres comprised at least in part of a biocompatible polyphosphazene polymer such as poly[bis(trifluoroethoxy)phosphazene] or derivatives thereof may be injected or otherwise delivered within the expandable semi-compliant structure as described above. Such microspheres may also contain bone cement and/or other active agents. In this and other aspects of this invention, disclosure that relates to using poly[bis(trifluoroethoxy)phosphazene] or other polyphosphazene polymers as a vehicle to deliver a drug or other active substance is applicable to all forms of the poly[bis(trifluoroethoxy)phosphazene] or other polyphosphazene polymer and all methods of using the poly[bis(trifluoroethoxy)phosphazene] or other polyphosphazene polymer, regardless of how it is fabricated or used in this invention. For example, FIG. 10 is a side elevational view of the detachable semi-compliant structure and catheter of FIG. 1 as expanded by a bone-supporting microspheres comprising a polyphosphazene polymer such as poly[bis(trifluoroethoxy)phosphazene]. The microspheres illustrated in FIG. 10 may also contain bone cement and/or other active agents.

In yet other preferred embodiments of the present invention, a biocompatible polyphosphazene polymer such as poly[bis(trifluoroethoxy)phosphazene] or derivatives thereof may be injected or otherwise delivered within the expandable semi-compliant structure as described above. Such a biocompatible polyphosphazene polymer may be used in the present invention in a gel or solid form.

In a preferred embodiment of the present invention, a biocompatible polyphosphazene polymer is provided in particulate form as microspheres. Such microspheres may also contain bone cement and/or other active agents. In this and other aspects of this invention, disclosure that relates to using poly[bis(trifluoroethoxy)phosphazene] or other polyphosphazene polymers as a vehicle to deliver a drug or other active substance is applicable to all forms of the poly[bis(trifluoroethoxy)phosphazene] or other polyphosphazene polymer and all methods of using the poly[bis(trifluoroethoxy)phosphazene] or other polyphosphazene polymer, regardless of how it is fabricated or used in this invention.

As described herein, “particle” and “particles” are used to mean a substantially spherical or ellipsoid article(s), hollow or solid, that may have any diameter suitable for use in the specific methods and applications described below, including a microparticle(s), a microsphere(s) and a nanosphere(s), beads and other bodies of a similar nature known in the art.

The preferred particles of the invention according to one embodiment described herein are composed, in whole or in part, the specific polyphosphazene polymer known as poly[bis(trifluoroethoxy) phosphazene] or a derivative of poly[bis(trifluoroethoxy) phosphazene]. Use of this specific polymer provides particles that are at least in part inorganic in that they include an inorganic polymer backbone and which are also biocompatible in that when introduced into a mammal (including humans and animals), they do not significantly induce a response of the specific or non-specific immune systems. The scope of the invention also includes the use(s) of such particles as controlled drug delivery vehicles.

The particles are useful in a variety of therapeutic and/or diagnostic procedures in part because, owing to the biocompatible nature of the polymer, the particles facilitate avoidance or elimination of immunogenic reactions generally encountered when foreign bodies are introduced into a mammalian body, such as “implant rejection” or “allergic shock,” and other adverse reactions of the immune system. Moreover, it has been found that the particles of the invention exhibit reduced biodegradation in vivo, thereby increasing the long-term stability of the particle in the biological environment. Moreover, in those situations where some degradation is undergone by the polymer in the particle, the products released from the degradation include only non-toxic concentrations of phosphorous, ammonia, and trifluoroethanol, which, advantageously, is known to promote anti-inflammatory responses when in contact with mammalian tissue.

In this aspect, each of the particles of the invention typically is formed at least in part of the polymer, poly [bis (2,2,2-trifluoroethoxy) phosphazene] or a derivative thereof. As described herein, the polymer poly[bis(2,2,2-trifluoroethoxy)phosphazene] has chemical and biological qualities that distinguish this polymer from other know polymers in general, and from other know polyphosphazenes in particular, The preferred poly[bis(trifluoroethoxy) phosphazene] polymer is made up of repeating monomers represented by the formula (I) shown below:

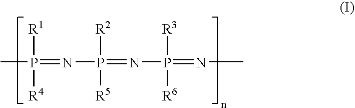

wherein R¹ to R⁶ are all trifluoroethoxy (OCH₂CF₃) groups and n may vary from at least about 100 to larger molecular weight lengths. For example, when R¹ to R⁶ are all trifluoroethoxy groups, n may be from about 4,000 to about 300,000, more preferably, n is about 5,000 to about 100,000 and most preferably n is about 13,000 to about 30,000. Additional ranges of n are provided in this disclosure, and additional substituents that can be represented by the groups R¹ to R⁶ are also provided herein.

It is preferred that the molecular weight of the polymer used to prepare the particles of the invention has a molecular weight based on the above formula, and more preferably, a molecular weight of at least about 70,000 g/mol, more preferably at least about 1,000,000 g/mol, and still more preferably a molecular weight of at least about 3×10⁶ g/mol to about 20×10⁶ g/mol. Most preferred are polymers having molecular weights of at least about 10,000,000 g/mol.

The diameter of a particle formed according to the invention will necessarily vary depending on the end application in which the particle is to be used. The diameter of such particles is preferably about 1 to about 5,000 μm, with a diameter of about 1 to about 1,000 μm being most preferred. Other preferred sizes include diameters of about 200 to about 500 μm, about 1 to about 200 μm and greater than about 500 μm. In methods using the particle where more than one particle is preferred it is not necessary that all particles are of the same diameter or shape.

The particles may also include other compounds which function to enhance, alter or otherwise modify the behavior of the polymer or particle either during its preparation or in its therapeutic and/or diagnostic use. For example, active agents such as peptides, proteins, hormones, carbohydrates, polysaccharides, nucleic acids, lipids, vitamins, steroids and organic or inorganic drugs may be incorporated into the particle. Excipients such as dextran, other sugars, polyethylene glycol, glucose, and various salts, including, for example, chitosan glutamate, may be included in the particle.

Additionally, if desired, polymers other than the poly[bis(trifluoroethoxy) phosphazene] and/or its derivative may be included with in the particle. Examples of polymers may include poly(lactic acid), poly(lactic-co-glycolic acid), poly(caprolactone), polycarbonates, polyamides, polyanhydrides, polyamino acids, polyorthoesters, polyacetals, polycyanoacrylates, and polyurethanes. Other polymers include polyacrylates, ethylene-vinyl acetate co-polymers, acyl substituted cellulose acetates and derivatives thereof, degradable or non-degradable polyurethanes, polystyrenes, polyvinylchloride, polyvinyl fluoride, poly(vinyl imidazole), chlorosulphonated polyolefins, and polyethylene oxide. One may incorporate the selected compounds by any means known in the art, including diffusing, inserting or entrapping the additional compounds in the matrix of an already formed particle or by adding the additional compound to a polymer melt or to a polymer solvent in the preparation of the particle such as described herein.

The loaded or unloaded particle may be coated with an additional polymer layer or layers, including polymers such as those mentioned hereinabove. Further, poly[bis(trifluoroethoxy) phosphazene or its derivatives may be used to form such a coating on a particle formed of other suitable polymers or copolymers known or to be developed in the art that are used to form particles as described herein. Preferably, when coating a particle such as a microparticle, poly[bis(trifluoroethoxy)phosphazene is applied as a coating on a microparticle(s) formed of an acrylic-based polymer as set forth in further detail below.

The microspheres may be prepared by any means known in the art that is suitable for the preparation of particles containing poly [bis(trifluoroethoxy)phosphazene]. In a procedure according to an embodiment herein a “polymer solution” is prepared by mixing one or more polymer solvent(s) and the poly[bis(trifluoroethoxy)phosphazene and/or a derivative thereof until the polymer is dissolved.

Suitable solvents for use in the preparation of the polymer solution include any in which the polymer poly[bis(trifluoroethoxy)phosphazene and/or its derivatives are soluble. Exemplary solvents include, without limitation, ethyl-, propyl-, butyl-, pentyl-, octylacetate, acetone, methylethylketone, methylpropylketone, methylisobutylketone, tetrahydrofirane, cyclohexanone, dimethylacetamide, acetonitrile, dimethyl ether, hexafluorobenzene or combinations thereof.

The polymer solution contains the poly[bis(trifluoroethoxy)phosphazene and/or its derivative polymer in a concentration of about 1% by weight of polymer to 20% by weight of polymer, preferably about 5% to 10% by weight of polymer. Other polymers, as discussed above, may be present in the solution, or may be added to the vessel in the form of a second solution powder or other form, if one wishes to include such polymers in the final particle.

In carrying out the process, the polymer solution is next dispensed, preferably in the form of drops or an aerosol, into a vessel containing a non-solvent. By “non-solvent” it is meant any organic or inorganic solvents that do not substantially dissolve the poly[bis(trifluoroethoxy)phosphazene polymer and which have a melting point that is lower relative to the melting point of the solvent in which the polymer is dissolved (“polymer solvent”), so that the non-solvent thaws before the solvent thaws in the course of the incubation step. Preferably, this difference between the melting point of the non-solvent and the polymer solvent is about 10° C., more preferably about 15° C., and most preferably, greater than about 20° C. Under certain conditions it has been found that the structural integrity of the resultant particle may be enhanced if the difference of the melting points of the polymer solvent and of the non-solvent is greater than 15° C. However, it is sufficient that the non-solvent point is merely slightly lower than that of the polymer solvent.

The non-solvent/polymer solvent combination is incubated for approximately 1 to 5 days or until the polymer solvent has been completely removed from the particles. While not wishing to be bound by theory, it is hypothesized that during the incubation, the non-solvent functions to extract the polymer solvent from the microscopic polymer solution droplets from the particles such that the polymer is at least gelled. As the incubation period passes, the droplets will shrink and the solvent becomes further extracted, leading to a hardened outer polymeric shell containing a gelled polymer core, and finally, after completion of the incubation, a complete removal of the residual solvent. To ensure that the polymeric droplets retain a substantially spherical shape during the incubation period, they are maintained in a frozen or substantially gelled state during most if not all of the incubation period. Therefore, the non-solvent temperature may stay below the melting point of the solvent during the cryoextraction process.

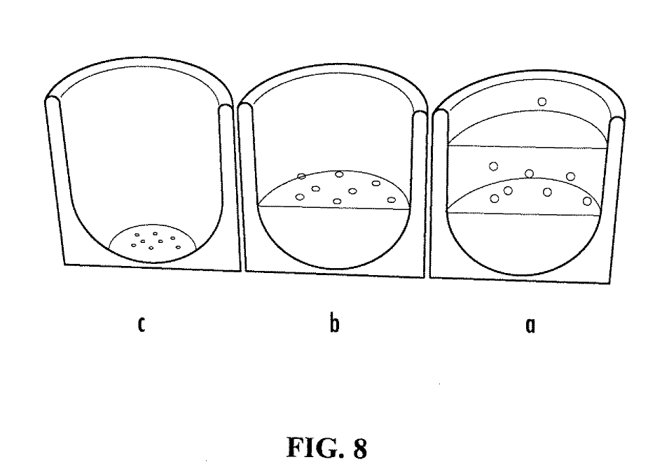

As shown in FIG. 8, at the vessel labeled (a), polymer solution droplets are shown being dispensed either with a syringe or other device at a controlled rate onto a top layer of liquid nitrogen. The nitrogen layer is situated over a bottom layer consisting of the selected non-solvent, which will eventually serve to extract the solvent from the frozen polymer solution droplets. The non-solvent layer has been previously frozen with liquid nitrogen prior to the dispensing of the polymer solution. The vessel labeled (b) shows the onset of the dewing of the frozen nonsolvent, into which the frozen polymeric droplets will sink. The vessel labeled (c) shows the cryoextraction procedure after approximately three days of incubation wherein the polymer solution droplets, incubated within the non-solvent, have been depleted of a substantial amount of solvent. The result is a gelled, polymeric particle in the form of a bead having a hardened outer shell. As can be seen by the representation, the non-solvent height within the vessel is slightly reduced due to some evaporation of the non-solvent. The size of the beads will shrink quite substantially during this process depending on the initial concentration of the polymer in the polymer solution.

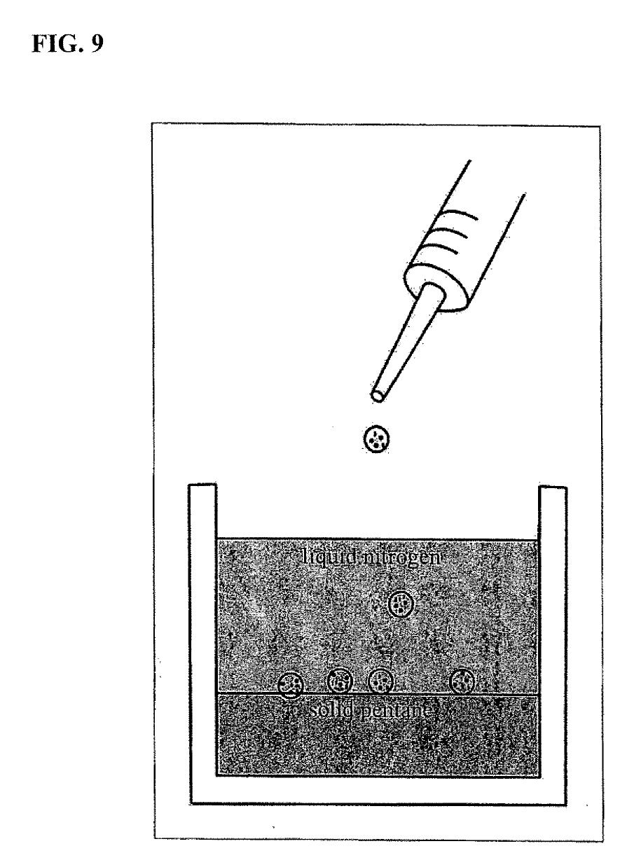

In one embodiment of a method of preparing a poly[bis(trifluoroethoxy)phosphazene-containing particle(s) according to the invention, such particles can be formed using any way known or to be developed in the art. Two exemplary preferred methods of accomplishing this include wherein (i) the non-solvent residing in the vessel in the method embodiment described above is cooled to close to its freezing point or to its freezing point prior to the addition of the polymer solution such that the polymer droplets freeze upon contact with the pre-cooled non-solvent; or (ii) the polymer droplets are frozen by contacting them with a liquefied gas such as nitrogen, which is placed over a bed of pre-frozen non-solvent (see, FIG. 9). In method (ii), after the nitrogen evaporates, the non-solvent slowly thaws and the microspheres in their frozen state will sink into the liquid, cold non-solvent where the extraction process (removal of the polymer solvent) will be carried out.

By modifying this general process, one may prepare particles that are hollow or substantially hollow or porous. For example, if the removal of the solvent from the bead is carried out quickly, for example, by applying a vacuum during the final stage of incubation, porous beads will result.

The particles of the invention can be prepared in any size desired, “Microspheres” may be obtained by nebulizing the polymer solution into a polymer aerosol using either pneumatic or ultrasonic nozzles, such as, for example a Sonotek 8700-60 ms or a Lechler US50 ultrasonic nozzle, each available from Sono[.tek] Corporation, Milton, N.Y., U.S.A. and Lechler GmbH, Metzingen, Germany. Larger particles may be obtained by dispensing the droplets into the non-solvent solution using a syringe or other drop-forming device. Moreover, as will be known to a person of skill in the art, the size of the particle may also be altered or modified by an increase or decrease of the initial concentration of the polymer in the polymer solution, as a higher concentration will lead to an increased sphere diameter.

In an alternative embodiment of the particles described herein, the particles can include a standard and/or a preferred core based on an acrylic polymer or copolymer with a shell of poly[bis(trifluoroethoxy)phosphazene. Such particles can provide a preferred spherical shape and improved specific gravity for use in a suspension of injection or delivery medium. The acrylic polymer based polymers with poly[bis(trifluoroethoxy)phosphazene shell described herein provide a substantially spherical shape, mechanical flexibility and compressibility, improved specific gravity properties. The core polymers may be formed using any acceptable technique known in the art, such as that described in B. Thanoo et al., “Preparation of Hydrogel Beads from Crosslinked Poly(Methyl Methacrylate) Microspheres by Alkaline Hydrolysis,” J. Appl. P. Sci., Vol. 38, 1153-1161 (1990), incorporated herein by reference with respect thereto. Such acrylic-based polymers are preferably formed by polymerizing unhydrolyzed precursors, including, without limitation, methyl acrylate (MA), methyl methacrylate (MMA), ethylmethacrylate (EMA), hexamethyl (HMMA) or hydroxyethyl methaerylate (HEMA), and derivatives, variants or copolymers of such acrylic acid derivatives. Most preferred is MMA. The polymer should be present in the core in a hydrated or partially hydrated (hydrogel) form. Such polymers are preferably cross-linked in order to provide suitable hydrogel properties and structure, such as enhanced non-biodegradability, and to help retain the mechanical stability of the polymer structure by resisting dissolution by water.

Preferably, the core prepolymers are formed by dispersion polymerization that may be of the suspension or emulsion polymerization type. Emulsion polymerization results in substantially spherical particles of about 10 nm to about 10 microns. Suspension polymerization results in similar particles but of larger sizes of about 50 to about 1200 microns.

Suspension polymerization may be initiated with a thermal initiator, which may be solubilized in the aqueous or, more preferably, monomer phase. Suitable initiators for use in the monomer phase composition include benzoyl peroxide, lauroyl peroxide or other similar peroxide-based initiators known or to be developed in the art, with the most preferred initiator being lauroyl peroxide. The initiator is preferably present in an amount of about 0.1 to about 5 percent by weight based on the weight of the monomer, more preferably about 0.3 to about 1 percent by weight based on the weight of the monomer. As noted above, a cross-linking co-monomer is preferred for use in forming the hydrated polymer. Suitable cross-linking co-monomers for use with the acrylic-based principle monomer(s) used in preparing a polymerized particle core, include various glycol-based materials such as ethylene glycol dimethacrylate (EGDMA), diethylene glycol dimethacrylate (DEGDMA) or most preferably, triethylene glycol dimethacrylate (TEGMDA). A chain transfer agent may also be provided if desired. Any suitable MA polymerization chain transfer agent may be used. In the preferred embodiment herein, dodecylmercaptane may be used as a chain transfer agent in amounts acceptable for the particular polymerization reaction.

The aqueous phase composition preferably includes a surfactant/dispersant as well as a complexing agent, and an optional buffer is necessary. Surfactants/dispersants should be compatible with the monomers used herein, including Cyanamer® 370M, polyacrylic acid and partially hydrolyzed polyvinyl alcohol surfactants such as 4/88, 26/88, 40/88. A dispersant should be present in an amount of about 0.1 to about 5 percent by weight based on the amount of water in the dispersion, more preferably about 0.2 to about 1 percent by weight based on the amount of water in the dispersion. An optional buffer solution may be used if needed to maintain adequate pH. A preferred buffer solution includes sodium phosphates (Na₂HPO₄/NaH₂PO₄). A suitable complexing agent is ethylene diamine tetraacetic acid (EDTA), which may be added to the aqueous phase in a concentration of from about 10 to about 40 ppm EDTA, and more preferably about 20 to about 30 ppm. It is preferred that in the aqueous phase composition, the monomer to water ratio is about 1:4 to about 1:6.

The polymerization should take place at about ambient conditions, preferably from about 60° C. to about 80° C. with a time to gelation of about one to two hours. Stirring at rates of 100 to 500 rpm is preferred for particle formation, with lower rates applying to larger sized particles and higher rates applying to smaller sized particles.

Once PMMA particles, such as microparticles, are formed, they are preferably subjected to hydrolysis conditions typical of those in the art, including use of about 1-10 molar excess of potassium hydroxide per mol of PMMA. Such potassium hydroxide is provided in a concentration of about 1-15% potassium hydroxide in ethylene glycol. The solution is then heated preferably at temperatures of about 150-185° C. for several hours. Alternatively, to minimize reactant amounts and cost, it is preferred that lesser amounts of potassium hydroxide be used which are less than about 5 molar excess of potassium hydroxide per mole of PMMA, more preferably about 3 molar excess or less. For such hydrolytic reactions, a concentration of about 10-15% potassium hydroxide in ethylene glycol is also preferably used, and more preferably about 14% to about 15%. It will be understood by one skilled in the art, that heating conditions at higher temperatures may be used to decrease overall reaction times. Reaction times may be varied depending on the overall diameter of the resultant particles. For example, the following conditions are able to provide particles having about 35% compressibility and desired stability: for diameters of about 200-300 μm, the solution should be heated for about 7.5 to about 8.5 hours; for diameters of about 300-355 μm, about 9.5 to about 10.5 hours; for diameters of about 355-400 μm, about 11.5 to about 12.5 hours; and for about 400-455 μm, about 13.5 to about 14.5 hours, and the like. The particle size can be adjusted using variations in the polymerization process, for example, by varying the stirring speed and the ratio of the monomer to the aqueous phase. Further, smaller sizes can be achieved by increasing surfactant/dispersant ratio.

Following hydrolysis, particles are separated from the reaction mixture and their pH may be adjusted to any range as suited for farther processing steps or intended uses. The pH of the particle core may be adjusted in from about 1.0 to about 9.4, preferably about 7.4 if intended for a physiological application. Since size, swelling ratio and elasticity of the hydrogel core material are dependent on pH value, the lower pH values may be used to have beneficial effects during drying to prevent particle agglomeration and/or structural damage. Particles are preferably sieved into different size fractions according to intended use. Drying of particles preferably occurs using any standard drying process, including use of an oven at a temperature of about 40°-80° C. for several hours up to about a day.

To provide desired surface properties to the hydrophilic hydrogel particles, in order to provide adhesion for receiving a poly[bis(trifluoroethoxy)phosphazene coating, the surface of the hydrogel may be subjected to treatment with any suitable ionic or non-ionic surfactant, such as tetraalkylammonium salts, polyalcohols and similar materials. A more permanent change in adhesion properties is brought about by rendering the surface of the particles hydrophobic by reaction of its polymethacrylic acid groups with a suitable reactant. Suitable reactants include, but are not limited to, hydrophobic alcohols, amides and carboxylic acid derivatives, more preferably they include halogenated alcohols such as trifluoroethanol. Such surface treatment also prevents delamination of the coating from the core once the coating is applied. Preferred surface treatments may include, without limitation, an initial treatment with thionyl chloride followed by reaction with trifluoroethanol. Alternatively, the surface may be treated by suspending the particles in a mixture of sulfuric acid and a hydrophobic alcohol, such as trifluoroethanol. Such treatments are preferred if the particles are to be coated in that they minimize any delamination of a coating.

Alternatively, and most preferably, the PMA core particles may be coated with a surface layer of and/or infused with barium sulfate. The barium sulfate is radiopaque and aids in visualization of the finished particles when in use. It also provides enhanced fluidization properties to the particles such that it reduces agglomeration especially during drying and allows for fluid bed coating of the PMA particles with an outer coating of poly[bis(trifluoroethoxy) phosphazene, thereby providing improved adhesion between a poly[bis(trifluoroethoxy) phosphazene outer core and a polymeric acrylate core particles. By allowing fluidization even when the core particles are swollen, barium sulfate also improves the overall coating and adhesion properties. By enabling the coating of the core particles even in a swollen state with poly[bis(trifluoroethoxy)phosphazene, barium sulfate also reduces the potential tendency of the poly[bis(trifluoroethoxy)phosphazene shells to crack or rupture in comparison with coating the particles in a dry state and then later exposing the particles to a suspension in which the core particles swell and exert force on the shell of poly[bis(trifluoroethoxy)phosphazene. A coating of barium sulfate on the core particles is preferably applied by adhesion of the barium sulfate in the form of an opaque coating on the hydrogel surface of the PMA beads. Barium sulfate can further assist in reducing electrostatic effects that limit particle size. By allowing for absorption of additional humidity, the barium sulfate tends to counteract the electrostatic effects.

Barium sulfate crystals adhering only loosely to the PMA particles may be covalently crosslinked or chemically grafted to the particle surface by spraycoating a sufficient amount of an aminosilane adhesion promoter onto the PMA particle. This will help to effectively reduce barium sulfate particulate matter in solution after hydration of the particles. Exemplary aminosilane adhesion promoters include, for example, 3-aminopropyl-trimethoxysilane, N-methyl-aza-2,2,4-trimethylsilacyclopentane, 2,2-dimethoxy-1,6-diaza-2-silacyclooctane, (3-trimethoxysilylpropyl)diethylene triamine, N-(3-(trimethoxysilyl)propyl)methanediamine, N¹,N²-bis(3-(trimethoxysilyl)propyl)ethane-1,2-diamine, 1,3,5-tris(3-(trimethoxysilyl)propyl)-1,3,5-triazinane-2-4-6-trione, and similar silane-based adhesion promoters.

A further alternative for improving visualization of microparticles made as noted herein include the absorption of a water soluble organic dye inside the hydrogel core particles. Exemplary dyes are preferably those FDA dyes approved for human use and which are known or to be developed for safe, non-toxic use in the body and which are capable of providing acceptable contrast. Organic dyes may include dyes such as D&C Violet no. 2 and others preferably approved for medical device uses, such as for contact lenses and resorbable sutures. Whereas barium sulfate operates as an inorganic filler and finely dispersed pigment that makes the particles visible by light diffraction due to small crystal size, the dyes when impregnated in the particles absorb the complementary part of the visible color spectrum.

Particles, including microparticles made in accordance with the foregoing process for forming a core hydrogel polymer are then coated with poly [bis (trifluoroethoxy) phosphazene or its derivatives. Any suitable coating process may be used, including solvent fluidized bed and/or spraying techniques. However, preferred results may be achieved using fluidized bed techniques in which the particles pass through an air stream and are coated through spraying while they spin within the air stream. The poly [bis (trifluoroethoxy) phosphazene or derivative polymer is provided in dilute solution for spraying to avoid clogging of the nozzle.

Exemplary solvents for use in such solutions include ethyl acetate, acetone, hexafluorbenzene, methyl ethyl ketone and similar solvents and mixtures and combinations thereof, most preferred is ethyl acetate alone or in combination with isoamyl acetate. Typical preferred concentrations include about 0.01 to about 0.3 weight percent poly [bis (trifluoroethoxy) phosphazene or its poly [bis (trifluoroethoxy) phosphazene derivative in solution, more preferably about 0.02 to 0.2 weight percent poly [bis (trifluoroethoxy) phosphazene, and most preferably about 0.075 to about 0.2 weight percent. It should be understood based on this disclosure that the type of hydrogel core can be varied as can the technique for coating a particle, however it is preferred that a core which is useful in the treatment techniques and applications described herein is formed and subsequently coated with poly[bis(trifluoroethoxy) phosphazene or its derivatives as described herein.

For use in embodiments of the present invention, particle density is preferably taken into consideration to ensure beneficial properties for particle delivery. Possible clogging of a catheter-based delivery system may occur if using a density-mismatched delivery medium. In addition, it is desirable to include a certain minimum amount of contrast agent in the delivery medium to achieve sufficient levels of fluoroscopic contrast during surgery. Currently, the polymethacrylate hydrogel density is between 1.05 g/cm³ and 1.10 g/cm³ depending on the equilibrium water content. The most common iodinated nonionic contrast agent media with 300 mg iodine per ml have densities of 1.32-1.34 g/cm³. As used herein, “buoyancy” refers to the ability of the particles to be substantially free floating in solution that occurs when the density of the particle is substantially the same as the medium in which it is suspended. Coated particles formed in accordance with the present invention as described herein can reach buoyancy when there is approximately 30% contrast agent in the delivery medium, however, such levels can be adjusted for such preferred use according to techniques described herein.

One method for increasing the density of the particles is by use of heavy water or deuterium oxide (D₂O). When heavy water is used to swell the particles, D₂O displaces H₂O, thereby increasing the weight of the particles for better dispersion and buoyancy levels. Typically this leads to the ability to add higher amounts of contrast agent of at least about 5% using such a technique. However, some equilibrating effect can occur over time when the particles are contacted with an aqueous solution of contrasting agent. Thus, it is preferred that when using D₂O for this purpose, either that suspension times are kept to a minimum or, more preferably, that the contrast agent be provided in a solution which also uses D₂O.

Alternatively, particles of pH 1 can be neutralized with cesium hydroxide and/or the final neutralized particles can be equilibrated with cesium chloride. Such compounds diffuse cesium into the particles, such that either the cesium salt of polymethacrylic acid is formed or polymethacrylic acid is diffused and thereby enriched with cesium chloride.

The cesium increases the density of the particles, thereby increasing the ability to add higher amounts of contrast agent. Typical buoyancy levels can be adjusted using the cesium technique such that about 45 to about 50% contrast agent may be added to the delivery medium as is desired. Cesium salts are non-toxic and render the particles visible using fluoroscopy. Cesium's atomic weight of 132.9 g/mol is slightly higher than that of iodine providing beneficial effects including increase in overall density and enhancement of X-ray contrast visibility even without a contrast agent. For certain cancer treatments where a radioactive isotope of cesium is desired, such active agent can be used as an alternative cesium source rendering the particles buoyant in an injectable solution as well as able to be used as an active treatment source.

The above-noted techniques for improving density of particles, such as microparticles for applications where density and/or buoyancy in solution are applicable properties may be applied in to the preferred particles described herein and/or may be applied for other similar particles. It should be understood that the disclosure is not limited to cesium and/or D₂O treatment of the preferred particles herein and that such techniques may have broader implications in other particles such as other acrylic-based hydrogels and other polymeric particles.

As noted above, barium sulfate may be used between the core particles and the preferred poly [bis (trifluoroethoxy) phosphazene coating or introduced into the interior of the core particles using any technique known or to be developed in the art. Also, organic dyes may similarly be included in the particle core. These materials, particularly the barium sulfate, also contribute to an increase in density as well as providing radiopacity. In addition to a general density increase as provided by the above-noted D₂O or cesium compounds, the barium sulfate allows this benefit even upon substantial and/or full hydration, allowing particles in suspension to remain isotonic. Thus, a barium sulfate powder coating can provide an inert precipitate having no effect on physiological osmolarity.

It should be understood, based on this disclosure, that the various buoyancy additives noted above can be used independently or in combination to provide the most beneficial effects for a given core particle and coating combination.

The invention also includes methods of delivering an active agent to a localized bone defect within the body of a mammal. The method includes contacting the localized area with at least one of the particles of the invention as described above, such that an effective amount of the active agent is released locally to the area. Diseases or pathologies that may be treated by this method include any wherein the localized or topical application of the active agent achieves some benefit in contrast to the systemic absorption of the drug. Suitable active agents include NSAIDS, steroids, hormones, nucleic acids, antibiotics, antiseptics, osteogenesis-enhancing agents, analgesics, anesthetics, or biological agents to promote regrowth of bone within the defect.

According to the present invention, one or more active agents may be delivered by using the hydrogel core of the microspheres as described herein as a delivery vehicle. Alternately or in conjunction with use of the hydrogel core as a drug delivery vehicle for active agents, active agents may also be delivered with microspheres of the present invention into defects in bone as a component of the injection or delivery medium, or instilled into such bone defects following delivery of the microspheres of the present invention.

As used herein, the terms “antiseptic agents” and “antiseptics,” which terms may be used interchangeably herein, are substances which may be used to reduce microbial levels and are biologically compatible enough to be applied to a particular anatomic surface without causing substantial irritation, inflammation, dysfunctional or other undesired reactions on or within the anatomic surface or adjacent tissues or organs. Antiseptic agents used in compositions according to the present invention may be microbicidal (bacteriocidal, fungicidal, and/or viricidal) in their actions, and are intended to provide a reduction in the ambient flora in the anatomic surface onto which they are administered.

As further used herein, the terms “antibiotic” and “antibiotics” are substances that are capable of destroying or weakening certain microorganisms, especially bacteria or fungi that may cause infections or infectious diseases. Antibiotics may be produced by or synthesized from other microorganisms, or they may be entirely synthetic compounds. Antibiotics may inhibit pathogenesis by interfering with essential intracellular processes, including the synthesis of bacterial proteins.

Examples of antibiotics and antiseptic agents as used herein include, but are not limited to, methylisothiazolone, thymol, .alpha.-terpineol, cetylpyridinium chloride, chloroxylenol, hexachlorophene, chlorhekidine and other cationic biguanides, methylene chloride, iodine and iodophores, triclosan, taurinamides, nitrofurantoin, methenamine, aldehydes, azylic acid, silver, benzyl peroxide, alcohols, carboxylic acids, salts, erythromycin, nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linexolid, temafloxacin, tosufloxacin, clinafloxacin, sulbactam, clavulanic acid, amphotericin B, fluconazole, itraconazole, ketoconazole, nystatin, penicilins, cephlalosporins, carbepenems, beta-lactams antibiotics, aminoglycosides, macrolides, lincosamides glycopeptides, tetracylines, chloramphenicol, quinolones, fucidines, sulfonamides, trimethoprims, rifamycins, oxalines, streptogramins, lipepeptides, ketolides, polyenes, azoles, and echinocandines. Other examples of antibiotics and antiseptics will readily suggest themselves to those of ordinary skill in the art.

As further used herein, the terms “anesthetic agents” and “anesthetics,” which terms may be used interchangeably herein, are substances which may be used to induce anesthesia, or reversibly depress neuronal function, producing total or partial loss of pain sensation when administered to an anatomic surface or tissue. Anestheics which may be used in the present invention include, but are not limited to, lidocaine, prilocaine, bupivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amid compounds such as dibucaine and related local anesthetic compounds; aminoketone compounds such as falicaine, dyclonine and related local anesthetic compounds; and amino ether compounds such as pramoxine, dimethisoquien, and related local anesthetic compounds, and derivatives, metabolites, and/or combinations thereof.

As further used herein, the terms “analgesic” and “analgesics” are pharmaceutical compounds capable of reducing or eliminating pain when administered to a mammalian patient. Analgesics which may be used in the present invention include, but are not limited to, morphine, fentanyl, sufentanil, remifentanil, and other opioids, tramadol, other non-opioid analgesics, and derivatives, metabolites, and/or combinations thereof.

Compositions of the present invention may comprise povidone-iodine as an antiseptic agent in a effective concentration of about 0.5%, about 1%, about 2.5%, about 5%, about 7.5%, about 10%, about 12.5%, about 15%, about 17.5%, and about 20%, where “about” means +/−1.25%.

As used herein, the term “biological agents” to promote regrowth of bone within the bone defect may comprise cells or fragments derived therefrom including, but are not limited to, bone marrow cells, mesenchymal cells, stromal cells, stem cells, embryonic stem cells, osteoblasts, precursor cells derived from adipose tissue, bone marrow derived progenitor cells, peripheral blood progenitor cells, stem cells isolated from adult tissue, and genetically transformed cells, or combinations of the above.

Alternatively, the microspheres of the present invention may be mixed with an inorganic filler. The inorganic filler may be selected from alphatricalcium phosphate, beta-tricalcium phosphate, calcium carbonate, barium carbonate, calcium sulfate, barium sulfate, hydroxyapatite, and mixtures thereof. In certain embodiments the inorganic filler comprises a polymorph of calcium phosphate. Such organic fillers according to the present invention may be incorporated in the hydrogel core of the microspheres, or delivered as a component of the injection/delivery medium used with the microspheres. In a preferred embodiment of the present invention, the inorganic filler is hydroxyapatite.

Embodiments of the present invention may further include bone adhesives in the injection or delivery medium, or such a bone adhesive may be instilled after delivery of the microspheres to the bone defect being treated. The term “bone adhesive” is used collectively herein to include bone glues and bone cements. The bone glues which can be used in the practice of the present invention include conventional biocompatible bone glues including 2-octyl cyanoacrylate and the like and equivalent thereof. The bone cements which can be used in the practice of the present invention include conventional biocompatible bone cements such as polymethylmethacrylate and the like. The bone glues and bone cements in the present invention may be absorbable or nonabsorbable.

If the particle formulated for delivery of an active agent to a localized area is about 1 to about 1,000 μm in diameter, the drug loaded microspheres can be applied to localized areas within the mammalian body using syringes and/or catheters as a delivery device, without causing inadvertent occlusions. As will be understood to a person of skill in the art, injection mediums include any pharmaceutically acceptable mediums that are known or to be developed in the art, such as, for example, saline, PBS or any other suitable physiological medium. In accordance with a further embodiment described herein, the invention includes an injectable dispersion including particles and a contrasting agent which particles are substantially dispersed in the solution. In a preferred embodiment, the particles are also detectible through fluoroscopy.

Moreover, it is envisioned that the active agent can be selected so as to complement the action of the particles in a synergistic fashion, especially if the particles are being used in an infectious or metaplastic application. For example, if the bone defect being treated is due to an osseous tumor, one may wish to load the particles used with a cytostatic drug, antiangiogenic agents, or an antimitotic drug. Alternately, if the bone defect being treated is due to an osteomyelitis, one may wish to load the particles used with one or more appropriate antibiotic agents.

In further alternative embodiments of the present invention, a coating of poly [bis (trifluoroethoxy) phosphazene may be applied to bioceramic microspheres, rather than to the hydrogel core previously described. In such embodiments, the bioceramic microspheres may be any biocompatible ceramic material including, but not limited to, hydroxyapatite ceramics, .beta.-tricalcium phosphate ceramics, biphasic calcium phosphate ceramics, macroporous ceramics, and calcium phosphate hydraulic cements.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of treating diseased or injured bone tissue comprising:
selecting an interior area in a bone tissue to be treated;
providing a device comprising a polyphosphazene;
inserting the device into the interior area of the bone tissue to be treated; and
internally supporting the bone tissue using the device during treatment;
wherein
the polyphosphazene has the formula: 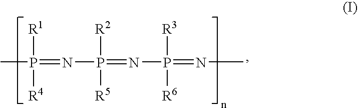
n is 2 to ∞; and
R¹ to R⁶ are each selected independently from alky, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof.

2. The method according to claim 1, wherein at least one of R¹ to R⁶ is an alkoxy group substituted with at least one fluorine atom.

3. The method according to claim 1, wherein R¹ to R⁶ are selected independently from OCH₃, OCH₂CH₃, OCH₂CH₂CH₃, OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF_{2,} OCH₂CH═CH₂, OCH₂CH₂CH═CH₂, or any combination thereof.

4. The method according to claim 1, wherein the polyphosphazene is poly[bis(2,2,2-trifluoroethoxy)]phosphazene or a derivative of poly[bis(2,2,2-trifluoroethoxy)]-phosphazene.

5. The method according to claim 1, wherein the device is a catheter having a structure capable of supporting the bone tissue, the catheter comprising an expandable three-dimensional, semi-compliant structure and fastener, and wherein the fastener releasably connects the catheter to the semi-compliant structure.

6. The method according to claim 5, wherein the catheter has a main body defining at least one interior passage therethrough, the semi-compliant structure defines an interior space, and the semi-compliant structure comprises a sealable port that allows for fluid communication between the interior passage of the catheter and the interior space of the semi-compliant structure.

7. The method according to claim 6, further comprising:
injecting a bone-supporting material into the semi-compliant structure through the passage within the catheter, through the sealable port, and into the interior space of the structure, thereby expanding the three-dimensional semi-compliant structure within the bone tissue;
wherein the bone-supporting material is selected from a liquid, a paste, a gel, a polymer, a cement, or any combination thereof.

8. The method according to claim 7, wherein the a bone-supporting material comprises poly[bis(2,2,2-trifluoroethoxy)]phosphazene or a derivative of poly[bis(2,2,2-trifluoroethoxy)]phosphazene.

9. The method according to claim 7, wherein the cement comprises a material selected from polymethylmethacrylate, dextran, polyethylene, carbon fiber, polyvinyl alcohol, or poly(ethylene terephthalate).

10. The method according to claim 7, wherein the hone-supporting material is capable of hardening to provide permanent bone support for the bone tissue.

11. The method according to claim 7, further comprising:
admixing a radiological treatment material with the bone-supporting material to form an admixture;
introducing the admixture into the semi-compliant structure, wherein the semi-compliant structure is capable of facilitating exposure of the area to be treated to the radiological material; and
exposing the bone tissue to the radiological treatment.

12. The method according to claim 11, wherein the radiological treatment is a time-release medication, wherein the medication is selected to exfiltrate the semi-compliant structure and coat the surface of the semi-compliant structure.

13. The method according to claim 11, wherein the semi-compliant structure comprises a semi-permeable material and the medication diffuses through the semi-permeable material to treat a disease or injury.

14. The method according to claim 13, wherein the semi-permeable material is selected from a polyphosphazene, a polyester, a polyethylene, a polylactic acid, a resorbable synthetic material, a suture material, a biocompatible Nitinol, or any combination thereof.

15. The method according to claim 13, wherein the semi-permeable material comprises poly[bis(2,2,2-trifluoroethoxy)]phosphazene, or a derivative of poly[bis(2,2,2-trifluoroethoxy)]phosphazene.

16. The method according to claim 13, wherein the biocompatible Nitinol further comprises a polymeric coating comprising poly[bis(2,2,2-trifluoroethoxy)]phosphazene, or a derivative of poly[bis(2,2,2-trifluoroethoxy)]phosphazene.

17. The method according to claim 11, wherein the disease or injury is selected from osteoporosis, osteoporotic fractured metaphyseal and epiphyseal bone, osteoporotic vertebral bodies, fractures of vertebral bodies due to tumors, round cell tumors, avascular necrosis of the epiphyses of long bones, avascular necrosis of the proximal femur, distal femur and/or proximal humerus, defects arising from endocrine conditions, metastatic tumors, or any combination thereof.

18. The method according to claim 8. wherein the semi-compliant structure is detachable from the catheter, and the method further comprises:
detaching the catheter from the semi-compliant structure;
sealing the sealable port; and
maintaining the bone-supporting material within the structure in a pressurized environment, thereby preventing the bone-supporting material from exuding from within the structure, to provide temporary support of the bone tissue.

19. The method according to claim 18, further comprising:
reattaching the catheter to the sealable port; and withdrawing the bone-supporting material.

20. A device for treating diseased or injured bone comprising:
a catheter, wherein the catheter comprises a main body defining at least one interior passage therethrough;
an expandable semi-compliant structure, wherein the semi-compliant structure defines an interior space;
a removable fastener, wherein the fastener removably connects the catheter to the semi-compliant structure; and
optionally, a sealable port, through which the interior passage of the catheter is in fluid communication with the interior space of the semi-compliant structure;
wherein at least one of the catheter, the expandable semi-compliant structure, or the removable fastener further comprises a polyphosphazene having the formula: 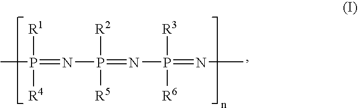
n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof.

21. The device according to claim 20, wherein at least one of R¹ to R⁶ is an alkoxy group substituted with at least one fluorine atom.

22. The device according to claim 20, wherein R¹ to R⁶ re selected independently from OCH₃, OCH₂CH₃, OCH₂CH₂CH₃, OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, OCH₂CH═CH₂, OCH₂CH₂CH═CH₂, or any combination thereof.

23. The device according to claim 20, wherein the polyphosphazene is poly[bis(2,2,2-trifluoroethoxy)]phosphazene or a derivative of poly[bis(2,2,2-trifluoroethoxy)]-phosphazene.

24. The device of claim 20, wherein the expandable semi-compliant structure comprises a polymeric coating comprising poly[bis(2,2,2-trifluoroethoxy)]phosphazene or a derivative of poly[bis(2,2,2-trifluoroethoxy)]phosphazene.

25. A device for treating diseased or injured bone comprising:
a catheter, wherein the catheter comprises a main body defining at least one interior passage therethrough;
an expandable semi-compliant structure, wherein the semi-compliant structure defines an interior space;
a screw device adapted to removably connect the catheter to the semi-compliant structure; and
optionally, a sealable port, through which the interior passage of the catheter is in fluid communication with the interior space of the semi-compliant structure, adapted to seal upon disconnecting the catheter from the semi-compliant structure;
wherein at least one of the catheter, the expandable semi-compliant structure, or the screw device comprises, or is coated with, poly[bis(2,2,2-trifluoroethoxy)]phosphazene.
